(19) United States
(12) Patent Application Publication
(10) Pub. No.: US 2013/0178933 A1
(43) Pub. Date: Jul. 11, 2013

(54) Ocular Device

(71) Applicants: Jose Javier Serrano Olmedo, Madrid (ES); Ruben Antonio Garcia Mendoza, Madrid (ES); Alejandra Mina Rosales, Madrid (ES); Diego Ruiz Casas, Madrid (ES); Diego Losado Bayo, Madrid (ES); Francisco Jose Munoz Negrete, Madrid (ES); Gema Rebolleda Fernandez, Madrid (ES); Alvaro Munoz Noval, Madrid (ES); Miguel Jose Manso Silvan, Madrid (ES); Raul Jose Martin Palma, Madrid (ES); Vicente Torres Costa, Madrid (ES)

(72) Inventors: Jose Javier Serrano Olmedo, Madrid (ES); Ruben Antonio Garcia Mendoza, Madrid (ES); Alejandra Mina Rosales, Madrid (ES); Diego Ruiz Casas, Madrid (ES); Diego Losado Bayo, Madrid (ES); Francisco Jose Munoz Negrete, Madrid (ES); Gema Rebolleda Fernandez, Madrid (ES); Alvaro Munoz Noval, Madrid (ES); Miguel Jose Manso Silvan, Madrid (ES); Raul Jose Martin Palma, Madrid (ES); Vicente Torres Costa, Madrid (ES)

(73) Assignees: UNIVERSIDAD POLITECNICA DE MADRID, Madrid (ES); FUNDACION PARA LA INVESTIGACION BIOMEDICA DEL HOSPITAL, MADRID (ES); (CIBER BBN), Zaragoza (ES); CENTRO DE INVESITACION BIOMEDICA EN RED EN BIOINGENIERIA, BIOMATERIALES Y NANOMEDICINA; UNIVERSIDAD AUTONOMOA DE MADRID, MADRID (ES)

(21) Appl. No.: 13/534,583

(22) Filed: Jun. 27, 2012

(30) Foreign Application Priority Data
Jul. 19, 2011 (ES) P201131226

(51) Int. Cl. A61F 2/14

(52) U.S. Cl. CPC A61F 2/14
USPC 623/4.1

(57) ABSTRACT

The present invention describes an ocular device comprising a magnetic eye implant and a ferrofluid. The products of the invention solve the problems of the patients' position and contribute to the increase in the success rate of the surgical procedure for correcting retinal detachment, problems of severe proliferative diabetic retinopathy in those incapable of maintaining posture, of infectious retinitis, problems resulting from traumatisms and endophthalmitis. It is also possible to use the device as an auxiliary surgical element, e.g., for extracting subretinal fluid.

### TECHNICAL FIELD

The present invention describes a device combining magnetic particles and a magnetic implant on sclera, useful as a retinal tamponade and for the postoperative treatment of eye surgery. The invention is comprised in the technical field of health technologies, more specifically in micro- and nanotechnologies, implants for human body and their biomedical applications specializing in ophthalmic biomedical applications. It applies to optimizing and aiding eye surgical procedures, as well as to improve the postoperative conditions of patients.

### STATE OF THE ART

The high incidence rate of retinal detachment (RD) in any of its forms ranks this human eye condition as one of the main targets of ophthalmologists. The pathology typically occurs due to the separation of the retinal pigment epithelium (EPR) layer from the rest of the neurosensory retina (RNS) leaving subretinal fluid (SRF) between them.

RD can be classified into three types according to its etiopathogenesis: exudative, tractional and rhegmatogenous. The latter is the most common and is caused by full thickness retinal breaks, retinal tears or holes.

There are several therapeutic modalities to close retinal breaks such as retinopexy by means of cryopexy and photocoagulation, pneumatic retinopexy, extraocular procedures with cerclage and implants and pars plana vitrectomy (PPV); the latter is the most commonly used procedure in treating RD today.

After a vitrectomy procedure the retina is reapplied by means of perfluorocarbon (PFC) liquids, liquid-air exchange or liquid-silicone exchange. The most common procedure for applying retina is the use of PFC, but these liquids cannot be left for too long inside the eye because they disperse into bubbles after 2-3 days reducing visual acuity (AV), they can move to the subretinal space, blocking the trabecular meshwork by increasing the intraocular pressure (IOP) and, in addition to their chemical toxicity, they may be toxic due to mechanical pressure as a result of the high specific gravity thereof damaging the outer layers of the retina.

Once the retina is applied and the PFC has been extracted retinopexy procedures such as cryopexy or endolaser photocoagulation which allow increasing the adhesivity of EPR and RNS by means of healing and gliosis are performed in order to prevent recurring RD and to assure long-term therapeutic success. With the retina applied and after the retinopexy procedure a retinal tamponade is left occupying the vitreous chamber. This tamponade can be gas, silicone or a mixture thereof, which allows keeping the retina applied until the retinopexy has taken its effect and blocking the retinal holes.

Gases are less dense than physiological saline filling the eyeball after vitrectomy, they therefore rise, and by means of the positioning of the patient's head and a posture maintenance favoring the position of the gas on the tear, they are directed to act as retinal tamponades. The force exerted on the retina is 10 times greater than with silicone.

The use of the gases as tamponades requires careful postoperative care with prone positioning to prevent pupillary block, cataract and endothelial damage, IOP control due to the risk of ocular hypertension (OHT), as well as posture maintenance so that the gas bubble rises blocking the area of the retinal breaks for long enough so that the retinopexy takes its effect. In addition to OHT, the possible complications derived from using gases as tamponades are cataract induction, endothelial decompensation and the passage of the gas to the subretinal space.

Silicones are used in the treatment of RD in especially complicated cases requiring long-term blocking. The silicones allow doing without a demanding postoperative period in the sense of posture and maintain a long-term blocking, but they also have significant side effects and complications since silicone can move to the suprachoroidal or subretinal space.

The prior art publications related with the invention include, for example US 2010074957 A1 describing a device for delivering intraocular drugs for the treatment of eye conditions with biodegradable microspheres containing active agents and a viscous carrier medium. However, these components are different from those of the present invention. Furthermore, the magnetic particles of the invention interact with the magnetic eye implant and they cannot be biodegradable due exactly to their durability requirement since they would not block the retinal hole. On the other hand, the use of a viscous medium for inserting the magnetic particles into the intraocular cavity is not essential in the present invention. Therefore in no aspect does this publication suggest the protected subject-matter of the present invention.

International publication WO 2009074823 A1 describes a pharmaceutical composition for the treatment of retinal detachment. The composition is based on nanoparticles with a specific gravity greater than that of the liquid allowing blocking and releasing the drug. However, they do not use the magnetic interaction of the nanoparticles nor do they use a magnetic eye implant with which they interact, such as in the present invention.

EP 1863420 A1 describes an ocular microimplant for the treatment of eye diseases. It comprises the homogenous mixture of an active agent in a biodegradable polymer matrix for releasing a drug introduced in the intraocular area. The present invention also uses a polymer to recover the eye implant, but it is a magnetic implant which is sutured to the sclera. The procedure and materials used are not even similar. Therefore, this publication also does not affect the patentability of the invention.

WO 2008044229 A1 uses a viscoelastic ferrofluid of nanometric particles with a specific viscosity range acting as lubricant, which determines the properties thereof. In the case of the present invention what is important is not the aqueous medium but the particle itself, and it allows adhering the retina to the choroids exerting the necessary pressure in a surgery of this type due to its micrometric size features, flake-type morphology and production. This means that the aqueous medium of the invention is reabsorbed, which does not occur with the ferrofluid of the publication since they would loss their lubricating properties. The authors of the publication themselves state that their system does not at all resemble ferrofluids for medical applications. Regarding implants, the publication describes an orthopedic type plate without any coating for intra-bone positioning. However, it is essential for implant of the present invention to have a good coating so that there will be no interaction with the living tissue and thus prevent the natural defensive reaction of the human body. Furthermore, in the magnetic implants of the present invention the sutures have special importance and require the presence of wedges to enable suturing to the sclera and offer stability to the ocular device, something not needed by the system of the publication because the bone already allows it to have an appropriate stability which does not require sutures. In conclusion, the publication of reference does not affect the inventive step of the present invention since it describes different designs, applications and uses.

Spanish publications ES 2024242 A6 and ES 2132029 B1 describe devices for operating on retinal detachment comprising magnets having an individual functional magnet design for focusing the magnetic field into a single retinal hole, because a separation which may cause interferences in the behavior of the counterpart of the system is proposed. However, the sizes of the particles and therefore their behavior are not comparable since the spheres described have a minimum size of 1 mm whereas the flake-type particles proposed in the present invention do not exceed 300 micron. The pressures exerted with spherical particles cannot be compared to the precision achieved with said flake-type particles, which are stacked such that the pressure they exert can be controlled to cause the least cell death possible. Furthermore, the vitrectomy proposed in the present invention is not invasive, whereas the sclerotomy methodology proposed by these publications is invasive. In conclusion, these publications also do not suggest the inventive aspects of the present invention.

Chinese publication CN 101524303 A describes a methodology for correcting retinal detachment which comprises injecting a liquid with magnetic particles interacting with a ring on the sclera, said ring containing a fluid with magnet powder. On the other hand, US 20050203333 A1 describes a procedure, materials and methods for correcting retinal detachment using a biomagnetic structure by means of photo-initiated polymerization for obtaining a polymer including ferromagnetic particles. It has the significant advantage of reducing the invasiveness of some surgical procedures such as retina reparation, as well as placing the biomagnetic components without needing suturing. The publication describes retinal detachment surgery in which a magnetized system and a polymer including magnetic particles is used. For retinal reparation, it describes a tamponade agent which successfully closes the retinal hole preventing the fissures produced on the sclera due to conventional buckle. It also mentions the possibility of using external magnets for manipulating nanomagnetic particles (paragraph 0023), which however does not suggest the use of an implant in external contact with the living tissue for positioning the magnetic fluid such as in the present invention. The publication further uses nanoparticles, whereas flake-type microparticles mostly having a size of 100 μm are used in the present invention. The possibility that the particles go through the retina into the bloodstream is thus prevented, a controlled pressure exerted on the retina being achieved.

That very publication US 20050203333 A1 which can be considered as the closet prior art, emphasizes the absence of suturing in all its elements, it therefore does not suggest the sutured implant of the present invention. It does suggest that magnets could be used for diffusing the nanoparticles; however the exact opposite is sought in the present invention: focusing the magnetic fluid in a specific point which is only achieved with a good design of the implant and microparticles used. Finally, the publication suggests the use of external magnets to move the magnetic fluid to the sclera from the inside, which does not apply in the present invention since what is desired is not to cross the retina but to block it. The publication mentions three particle diameters of 2, 4 and 8 mm (MagnaQuench), it describes how the magnetic fields achieved by these magnets are too strong to block the retinal hole and concludes that there is no solution for controlling the magnetic field other than considering the amount of magnetized particles, i.e., their concentration in scleral buckle. Referring to the magnetic fluid, it constantly mentions the use of magnetic particles cloistered in silicone for better manipulation, therefore the use thereof in vitreous humour or physiological saline such as in the present invention is not suggested. In the invention the blocking is performed without elements assisting the magnetic particles. Unlike the present invention, it also does not describe the use of pars plana vitrectomy.

With reference to the magnetic ocular implants, none of the two publications suggested the use of NdFeB magnets with Ni—Cu—Ni coating as an eye implant, nor do they described the two outermost layers described in the present invention preventing the interaction between the implant and the living tissue. On the other hand, the ferrofluid of spherical nanoparticles used in these publications do not suggest the use of magnetic flake-type particles which, due to their morphological, production, size and composition characteristics, allow putting the retina on the choroids causing a controlled pressure.

The purpose of the two preceding publications is to treat retinal detachment using magnetic force interacting with nanomagnetic particles immersed in a viscous medium. However, none of them use a magnetic eye implant or a ferrofluid such as that of the present invention. In view of the foregoing, none of the patentability of the embodiments of the invention is affected.

Therefore, the problem set out by the prior art is how to achieve an efficient system for blocking the retinal hole which improves the postoperative treatment in patients undergoing retina operations. The solution provided by the present invention is a device comprising a magnetic implant and a ferrofluid interacting to keep the wound closed minimizing the incidence in the wellbeing of the patient of said operations.

### DESCRIPTION OF THE INVENTION

The present invention is an ocular device comprising at least one magnetic eye implant which in turn comprises a flat rare earth magnet with magnetization energy between 27,852.05 and 35,809.78 TA/m, at least one layer of nickel-copper-nickel (Ni—Cu—Ni) coating and another layer of epoxy or silicone elastomer coating on both faces, and a ferrofluid which in turn comprises a colloidal suspension of microparticles, in which said ferrofluid and said magnetic implant eye interact.

The rare earth magnet is preferably an NdFeB (neodymium-iron-boron) magnet. In another preferred embodiment, the epoxy or silicone elastomer coating is of a thickness comprised between 1 and 1000 μm, more preferably between 1 and 500 μm.

In the present application “magnetic implant” is understood as a device which is sutured to the sclera and which acts like a magnet, but the magnetic material of which does not interact with the living tissue.

In the present application “ferrofluid” is understood as a solution formed by magnetic microparticles in colloidal suspension in a mono- or polydisperse liquid.

A preferred embodiment of the device of the invention comprises a third layer of silica coating of the eye implant, preferably biofunctionalized with amino groups, with a thickness comprised between 0.001 and 25 μm on both faces. The active face of the magnetic eye implant is the surface of the magnet closest to the sclera, directly interacting with the ferrofluid delivered to the eyeball. To maximize the magnetic field of interaction with the ferrofluid, this active face of the implant must have a smaller coating thickness than the opposite face.

In the present application, “biofunctionalization” is understood as the process performed so that the elements adhered to the silica coating provide biochemical characteristics which allow conjugating the surface with molecules of interest, e.g., medicinal products, enzymes, etc.

The layer of silica and the amino groups are deposited on a thin sheet by plasma-enhanced chemical vapor deposition (CVD) techniques. To that end, the magnets are introduced in a plasma reactor activated by radiofrequency. A metal-organic precursor, for example aminopropyl triethoxysilane (APTS), is introduced therein together with the argon generated by the plasma, in a process which requires controlling the entrance flows of said argon and precursor, and the strength of the radiofrequency wave for obtaining the silica sheet.

The implant used in the invention has the advantage that the part of the coating is made in two phases which do not require incorporating silicones or derivates, and a better encapsulation is thus achieved.

Another embodiment is the ocular device of the invention in which the magnetic flow in the center of the faces of said implant is comprised between ±1 mT and ±500 mT.

A preferred embodiment of the invention is that said implant is cylindrical-shaped with dimensions of between 3 to 7 mm in diameter by 0.4 to 1.4 mm in height, preferably between 3 and 6 mm in diameter by 0.5 to 1 mm in height. Another preferred embodiment is that the magnetization of the implant is localized on the central axis of the cylinder and is comprised in a range of ±185 and ±195 mT, and another embodiment is that the magnetization of said implant is diametrical and is comprised in a range of ±1 and ±15 mT.

The magnetic ocular implants used in the present invention allow various configurations since they can be manipulated with ease to form square matrices, lines of two or more implants, etc., due to their magnetic properties, shape and biocompatibility. Furthermore, they can be placed such that their total magnetic response focuses in an area where the retinal hole is of greater proportions or is very localized.

The cover of the magnetic eye implant of the invention is designed to facilitate being sutured to the sclera as a result of the incorporation of four suture wedges (Cs).

In terms of the ferrofluid used in the invention, in a preferred embodiment it has a magnetic response between 10⁻⁶ and 10⁻⁴ Am²/g, which optimizes its interaction with the magnetic eye implant.

The ferrofluid of the invention can be dissolved in any solvent which aids the desired purposes, such that in another embodiment, the colloidal suspension of the ferrofluid is in physiological saline, vitreous humour or in an alcohol.

In another embodiment, the microparticles of the ferrofluid comprise porous silicon and ferrous material, or their oxide derivatives. In yet another preferred embodiment, said microparticles comprise a silicon base with magnetite particle incrustations, in the form of flake. Porous silicon flakes are formed with nanoparticles of ferrous material incrusted therein and on the surface thereof.

In the present application, “flake-type particle” is understood as a lenticular-shaped particle resembling a scale, the layout dimensions of which are of the order of 1 to 300 micron in length and of 1 to 10 micron in section.

In a preferred embodiment, said microparticles in the form of flake have a size between 1 and 300 μm and a concentration of between 10 and 250 mg/ml in the ferrofluid; and in another preferred embodiment, between 45 and 55% of said microparticles in the form of flake have a size of 100 μm. In another preferred embodiment, they are carriers of pharmacologically acceptable compounds which would aid therapy and eye surgery.

The main advantage of these flake-type particles lies in the fact that this design allows placing them flatly offering exceptional pressure conditions on the retinal surface. The range of pressures obtained is controlled by the magnetic interactions between the ferrofluid and implant and comprises between 0.05 mmHg and 2 mmHg.

Another preferred embodiment is that the microparticles used in the invention are essentially spherical magnetic microparticles with a core of ferrous material and silicon coating with a diameter of 2 μm, and a concentration of 200mg/ml.

Other different embodiment is that the microparticles used are essentially spherical magnetic microparticles of porous silicon with nanoparticle incrustations with a diameter between 6 μm and 10 μm, preferably 8 μm.

The flakes have a more complex interaction with the magnetic fields applied since they are reoriented and placed to have a greater contact surface. The spherical microparticles interact with the magnetic fields applied forming agglomerations, whereby a contact surface with minimum spaces between particles will be formed. With both types of components, the ferrofluid is capable of blocking either one or several retinal holes. On the other hand, both the flakes and the spherical microparticles used in the invention are biocompatible without adverse effects on the organism.

The ferrofluid used in the invention is used as a tamponade in the treatment of RD after completing vitrectomy. These intraocularly injected particles directed by a magnetic field created by a magnet sutured to the sclera, or the outer wall of the eye, in the area corresponding to the tears act as tamponades of the retinal holes causing the RD. The patient undergoes a much more bearable postoperative condition by allowing comfortable head-down postures and with good visual acuity. Furthermore, they prevent the optical effect of gases and silicones filling the eye in other treatments, and also side effects such as cataract and inflammation as well as the recurrence of the pathology.

The ferrofluid can be inserted using the pars plana vitrectomy techniques. In the present application vitrectomy or “pars plana vitrectomy” is understood as the surgical methodology used for correcting retinal detachment including placing several entries into the eyeball through the pars plana, which is the part of the eye where the thin layer of the retina is so adhered to the pigment epithelium that it makes detaching it by handling difficult. The ferrofluid will be positioned in the ocular cavity in the area of interest right in the counterpart where the magnetic eye implant has been placed and it must block the retinal hole.

The invention allows using between 1 and 100 μl of ferrofluid together with one or several magnetic ocular implants in several configurations, as many as there are retinal holes or variations of the magnetic flow densities are needed for blocking said holes using pars plana vitrectomy.

The placement of the implant or the magnetic ocular implants will depend on the needs of the medical specialists, either due to the existence of multiple detachments or a detachment of greater magnitudes requiring several configurations of the magnetic ocular implants.

The interaction of the ferrofluid is due to the magnetic fields imposed with the magnetic eye implant on the region of interest. The lines of magnetic field cause the magnetic particles to rearrange and agglomerate forming a tamponade base of the retinal holes. The confinement of the microparticles under the scleral implant prevents component dispersion inside the eye since they are permanently attracted by the field of the magnetic implant, exerting pressure on the inner limiting membrane against the choroids and the sclera located immediately below the magnetic scleral implant. After applying endolaser in the area of retinal detachment, the ferrofluid allows the suitable healing of said holes. The only restriction for the patient would be to remain exposed to intense external magnetic fields. The microparticles adhered like a film on the ocular wall on the retina in the inner limiting membrane, thereby leaving the visual axis free and the patient can see without refractive effects right after surgery.

The device of the invention offers compatibility with the use of a magnetic indenter which allows sucking the subretinal or subchoroidal space to drain blood, PFCL, air, silicone, exudates and other components which may be extirpated.

The magnetic microparticle complexes vs magnetic scleral implant of the invention can be used for retinal detachment both in vitrectomy procedure and without vitrectomy procedure, associating them to scleral implant surgery or pneumatic retinopexy.

Therefore, another embodiment is the use of the ocular device of the invention for the preparation of a surgical system useful for the operative treatment of pathologies associated with retina, preferably for retinal detachment.

### BRIEF DESCRIPTION OF THE DRAWINGS

FIG. 1.a shows a diagram of the approximate morphology of a microparticle in the form of flake.

FIG. 1.b shows a process for obtaining the flakes formed by a matrix of porous silicon with magnetite incrustations. Based on the starting crystalline silicon wafer electrochemical anodization in HF (1) is performed for forming multilayered porous silicon. Then an immersion is performed in a solution of nanoparticles of ferrous material (2) for depositing said nanoparticles on the surface and inside the matrix of porous silicon. A heat treatment (3) follows, and the removal of the layer of porous silicon containing ferrous material (4), followed by mechanical and/or ultrasonic grinding.

FIG. 2.a shows a schematic drawing of the design of the magnetic ocular implants of the invention, the magnetization of which is oriented on the central axis of the implant, i.e., the upper part is positive and the lower part is negative. The shape is a cylinder with a diameter (D) and a height (h).

FIG. 2.b shows a schematic drawing of the design of the magnetic ocular implants where (1) is the NdFeB magnet with magnetization in the centre of the cylinder and (2) the coating, either an epoxy or a silicone elastomer coating. The NdFeB magnet is cylindrical-shaped with a diameter (D) and a height (h). The coating is placed enveloping the magnet leaving a protective layer on both faces of the magnet (R1 and R2). It also shows the existence of a coating extension for placing the suture wedges (Cs).

FIG. 2.c shows a schematic drawing of the design of the magnetic ocular implants of the invention the magnetization of which is oriented on the diameter of the implant; i.e., positive from the center to the left side and negative towards the opposite side. The shape is a cylinder with a diameter (D) and a height (h).

FIG. 2.d shows a schematic drawing of the design of the magnetic ocular implants where (1) is the magnet of the invention with magnetization on the diameter of the cylinder and (2) the coating, either an epoxy or a silicone elastomer coating. The NdFeB magnet is cylindrical-shaped with a diameter (D) and a height (h). The coating is placed enveloping the magnet leaving a protective layer on both faces of the magnet (R1 and R2). It also shows the existence of a coating extension for placing the suture wedges (Cs).

FIG. 2.e shows a top view of the design of the magnetic eye implant. (1) is the magnet of the invention, with magnetization either on the diameter or on the central axis of the cylinder, and (2) the coating, either an epoxy or a silicone elastomer coating. The NdFeB magnet is cylindrical-shaped with a diameter (D) and the coating is placed enveloping the magnet leaving a protective layer and two coating extensions for placing four suture wedges (Cs). Said extensions protrude outwardly from the magnetic eye implant by a distance (Gs) between 1 and 3 mm and its height corresponds to that of the NdFeB magnets (h).

FIG. 3 shows three positioning examples of the magnetic ocular implants. The numbering corresponds to: (1) Sclera, (2) Choroids, (3) Retina, (4) Ferrofluid and (5) magnetic eye implant. In all the examples shown, a difference is made among (1), (2) and (3) since they will be affected by the magnetic interaction between (4) and (5). Example “A” refers to the blocking of a single retinal hole in the lower part of the eyeball making use of the magnetic interaction between a single dose of (4) and a piece of (5), whereby the conditions for blocking are complied with. Example “B” also refers to the blocking of a single retinal hole but for which it is necessary to focus the magnetic fields in the area of interest. For said purpose a single dose of (4) and two pieces of (5) are used. Example “C” refers to a multiple blocking of retinal holes for which two doses of (4) and three pieces of (5) are used, whereby it is possible to manipulate the magnetic fields and direct them so that the blocking is successful. The distance between the necessary elements, (4) and (5), is approximately 2 mm due to the presence of (1), (2) and (3).

FIG. 4 shows the diagram depicting the way of inserting and placing the magnetic extractor for removing the ferrofluid from the ocular cavity. The magnetic extractor is introduced in the ocular cavity through (1), (2) and (3) representing the sclera, choroids and retina, respectively. Since both (4) and (5) must be extracted simultaneously, the magnetic extractor made up of three parts: manipulation handle (6), magnetic tip (7), and extraction duct (8), is introduced. When (5) is removed, (4) remains loose inside the eye, to prevent this situation the extractor is placed close to (4) and (7) and (8) are activated to perform the complete extraction of (4).

### EXAMPLES

The following examples are provided to present the present invention in an illustrative but non-limiting manner.

### Example 1

### Obtaining the Implant

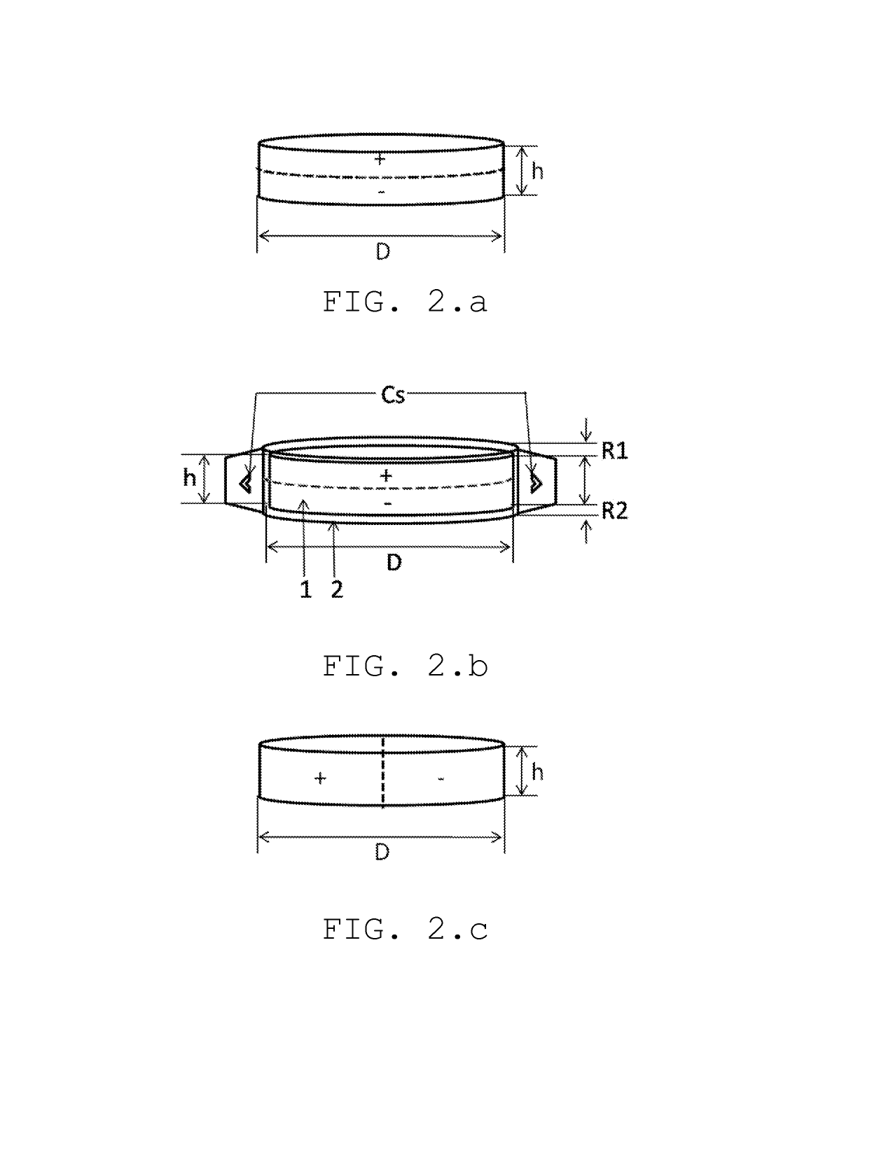

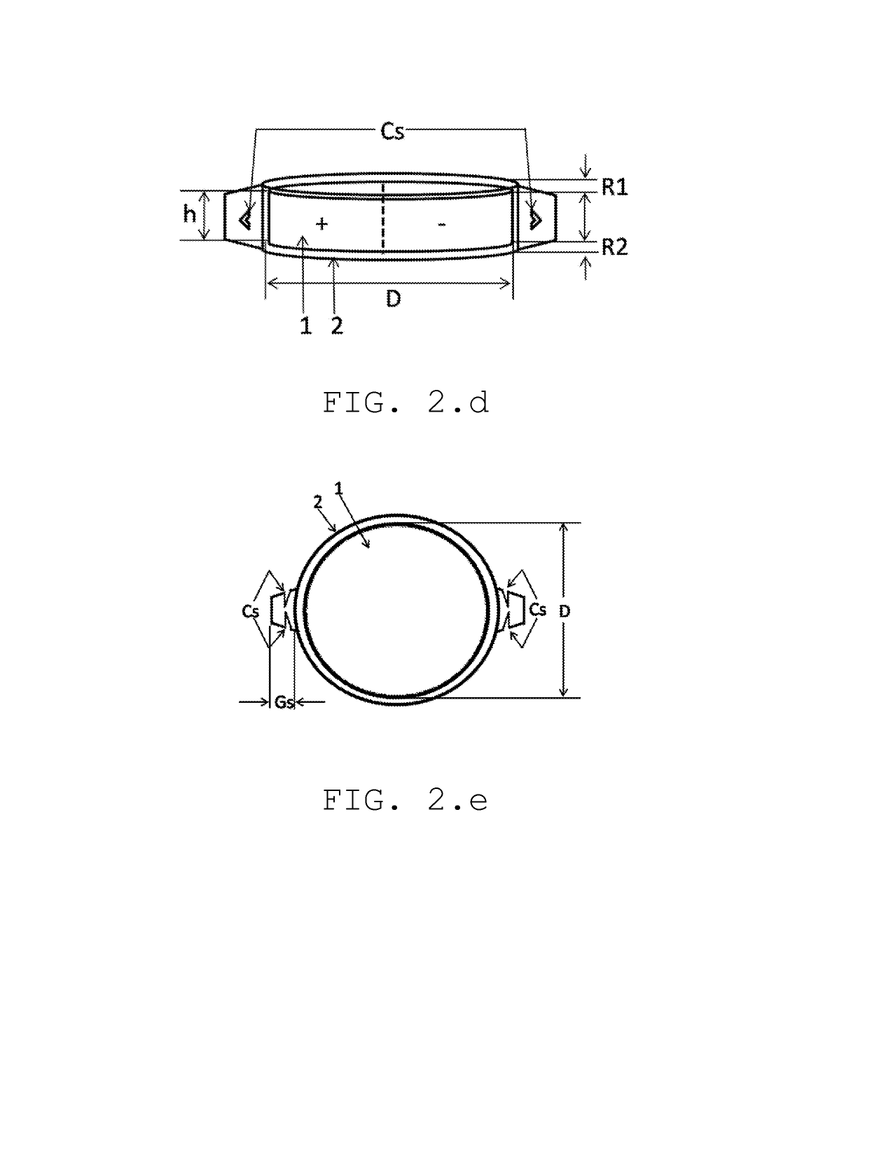

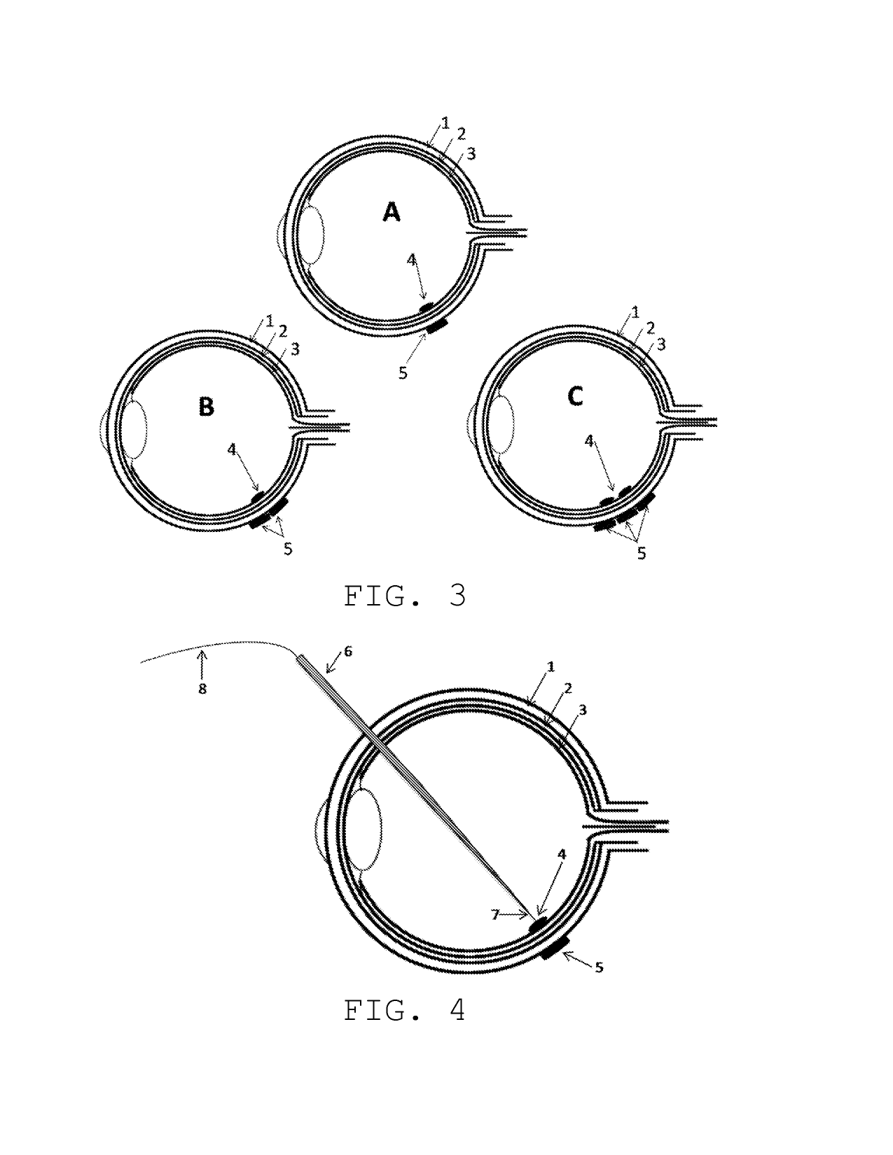

The magnetic eye implant had, as a core, a flat cylindrical NdFeB magnet with dimensions of 6 mm in diameter by 1 mm in height, with an associated coating made of Ni—Cu—Ni and magnetized along the central axis. It was coated with epoxy resin to eliminate its possible toxicity. Prior to preparing the epoxy, both the magnets and the molds were sterilized for 35 min in a class II biological safety cabin with a wavelength of 300 nm. 100 g of epoxy EPOFER EX 401 and 32 g of curing liquid EPOFER E 432 (FEROCAST) were also prepared and mixed until obtaining a homogenous and viscous solution. Once 35 minutes of sterilization elapsed, a layer of 1mm in height of the epoxy preparation was applied inside the mold in the cabin, it was left to settle for 20 min and 30 magnets were placed. Once the magnets were placed in the epoxy, it was left to settle for 18 h. After that time, epoxy was prepared again for the missing layer following the same procedure above. Once prepared, 1mm in height was added on the existing epoxy base to cover the magnets completely. They were left to settle for 18 h and extracted from the sterilization cabin to take them out of the mold and to give them the shape of FIGS. 2b, 2d and 2e including the wedges. The coated magnets were placed for 35 min in the sterilization cabin, together with the deposits where they were transferred to the following coating. This procedure resulted in an implant with an epoxy coating of between 0.3 and 0.5 mm on each face, and of the same magnitudes in their circumference, except the area of the wedges which has between 0.5 and 2 mm. The next silica and amino group coating was deposited in a thin sheet by plasma-enhanced chemical vapor deposition (CVD) techniques. To that end the magnets were introduced in a plasma reactor activated by radiofrequency, together with aminopropyl triethoxysilane (APTS) as the precursor and together with argon gas generated by the plasma. As indicated previously, by controlling the strength of the radiofrequency wave and the entrance flows of argon and precursor a thin silica sheet with amino ends was obtained. Finally a last sterilization process was performed in the class II biological safety cabin with a wavelength of 300 nm both on the end implants and on the deposits where they were kept until the placement thereof in the eye. Coated magnetic ocular implants for preventing the interaction with living tissue and for preventing toxicity, and therefore preventing rejection by the eye, were thus obtained.

### Example 2

### Sterilizing the Implant

Once the implant and the ferrofluid were obtained, they were introduced in a class II biological safety cabin with a wavelength of 300 nm. The elements were kept separately for 35 min under exposure to ultraviolet rays and for another 35 min once they are combined.

### Example 3

### Obtaining Flakes

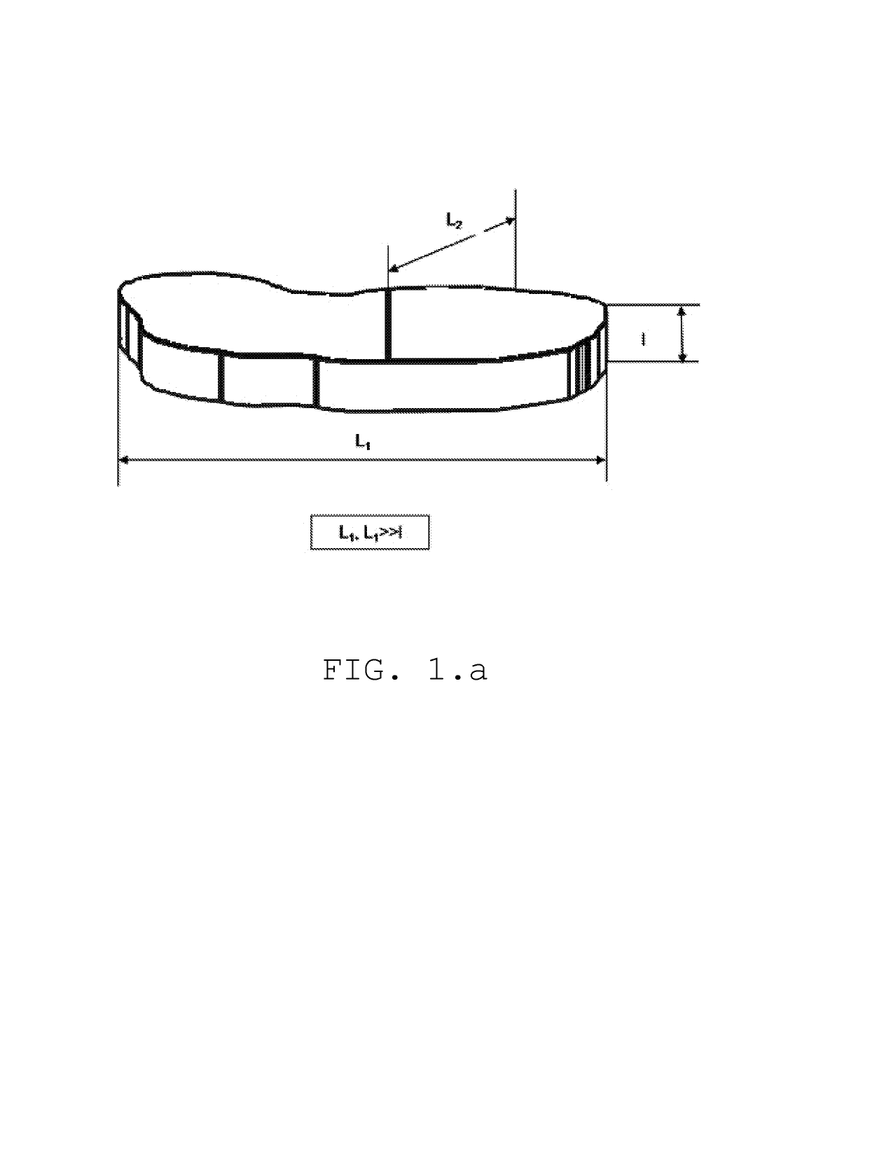

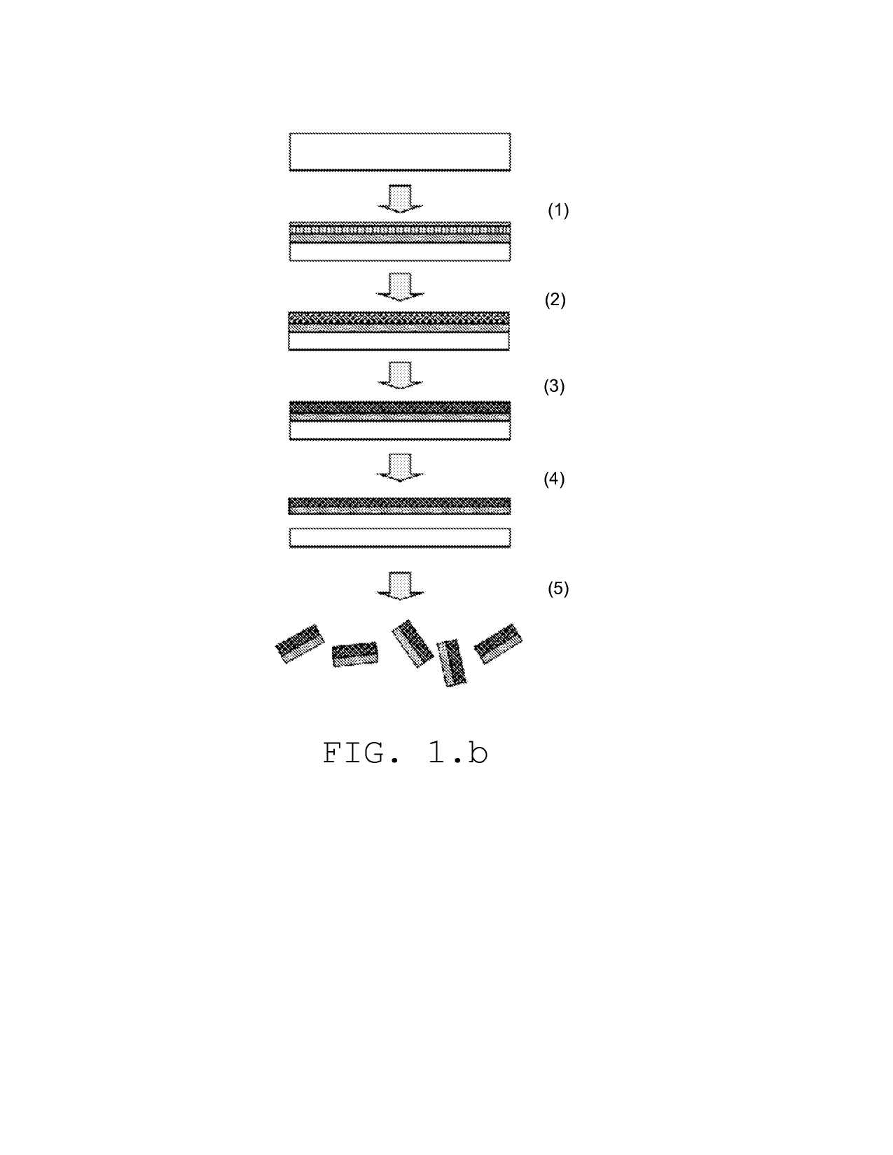

A doped p-type crystalline silicon wafer having a high conductivity, orientation 100 is used, which is previously metalized with aluminium on one of the faces by physical vapor deposition methods (MATTOX Donald M. Handbook of physical vapor deposition (PVD) processing (2nd Ed.), Noyes Publications (1998), Noyes Publications ISBN 0-8155-1422-0 and Mahan, John AND. Physical Vapor Deposition of Thin Films. New York: John Wiley & Sons, 2000. ISBN 0471330019), as the starting material and substrate. Anodization by electrochemical etching is performed on said substrate in a HF:ethanol solution for obtaining the porous multiplayer by means of a pulse of 100 mA/cm2 for 20 seconds, followed by a step in which a second innermost layer was formed with a 200 s-long pulse of 150 mA/cm2, which provides a suitable size to the pores in which the nanoparticles will be housed and aids in anchoring the layer of ferrous material. A pulse is then applied under low current: 80 mA/cm2 for 400 s for providing structural stability to the flake. Finally, a current of 200 mA/cm2 is applied for 10 s for forming a sacrificial layer which allows raising the multilayered porous silicon completely in the last phase of the preparation. The multilayer formed on the silicon substrate is then submerged in a 5 mg/ml solution of nanomagnetite particles having a size between 5 and 15 nm by means of the “Dip Coating” technique, after which it was left to dry. The immersion processes was repeated 3 times. Then, once the surface is dry, the samples are introduced in the oven for 2 h at 250° C. to eliminate the rests of the solvent and to favor the matrix and nanoparticles conjugation into a compact structure(FIG. 1.b).

### Example 4

### Obtaining the Ferrofluid with Flakes

Once the flakes according to Example 3 were obtained, the solvent and solute were conjugated for obtaining the ferrofluid. For such purpose, 50 mg of flakes were weighed and dissolved in 1 ml of physiological saline for obtaining a ferrofluid with a concentration of 50 mg/ml, all this inside a class II biological safety cabin with a wavelength of 300 nm. Once introduced, the flakes were subjected to a ultrasound bath with frequencies between 25 and 130 KHz for dissolving them more homogenously. Finally, the solution was sterilized in a class II biological safety cabin with a wavelength of 300 nm for a period of 30 min.

### Example 5

### Obtaining the Ferrofluid with Spherical Particles

Like Example 3, after obtaining the layer of porous silicon by means of a pulse of between 80 and 120 mA/cm2 and of 200 to 1000 s followed by a short pulse of 150 mA/cm2 and 10 s, the layer is extracted by water immersion. After extracting the complete layer, it is subjected to sonication to fragment it into what will be the micrometric sized spherical particles of porous silicon. The colloid is left to dry and an amount of a commercial nanomagnetic particle solution (magnetite, 5-15 nm, 5 g/l, SigmaAldrich) equivalent to the same value by weight of silicon-nanoparticles is added. The colloid is subjected again to sonication. The colloid is left to dry and the procedure is repeated up to 3 times. In the last cycle, the colloid is left to dry in a recipient with a wide opening and is introduced in the oven at 200° C., 2 h, microparticles of porous silicon and incrustations of ferrous material thus being obtained. 50 mg of these microparticles were weighed and dissolved in 1 ml of physiological saline for obtaining a ferrofluid with a concentration of 50 mg/ml, inside a class II biological safety cabin with a wavelength of 300 nm. Once introduced, the spherical particles were subjected to a ultrasound bath with frequencies between 25 and 130 KHz for dissolving them more homogenously. Finally, the solution was sterilized in a class II biological safety cabin with a wavelength of 300 nm for a period of 30 min.

### Example 6

### Blocking a Lateral Chamber Retinal Hole

50 μl of a ferrofluid with a concentration of 50 mg/ml of flakes with a size between 20 μm and 300 μm, mostly of 100 μm, having a silica matrix and magnetite incrustations and diluted in physiological saline were used. A magnetic eye implant of NdFeB with 42 MGOe, epoxy coating with a diameter of 6 mm and a thickness of 2 mm of the magnet and coatings together was also used. This implant was placed and sutured on the sclera of a rabbit in the lateral area of the ocular chamber where the retinal hole was found. A 25 G syringe in which 50 μl of ferrofluid were placed was used for inserting the ferrofluid. Making use of the orifices made in the pars plana vitrectomy surgical methodology the syringe was introduced in the eyeball and positioned on the retinal hole, in this case a lateral chamber retinal hole, for releasing the ferrofluid. Once released, the ferrofluid interacted with the magnetic eye implant sutured in advanced repositioning the retina on the choroids and blocking the hole. The ferrofluid and the magnetic eye implant were removed after 1 week. The results showed 90% of success in the re-application of retina at the end and no strange behavior which allows identifying post-operative recovery anomalies has been identified. The patient experiences no complications.

### Example 7

### Blocking of Multiple Holes Retinal

50 μl of ferrofluid with a concentration of 50 mg/ml of flake-type microparticles, with a size between 20 μm and 300 μm, mostly of 100 μm, silicon base and magnetite infiltrations were used. The particles were diluted in physiological saline. A number of magnetic ocular implants equal to the number of identified retinal holes, which in this were two, was used, all the implants are NdFeB implants with 42 MGOe with an epoxy coating, diameter of 6 mm and thickness of 2 mm all together between magnet and coatings. The implants were placed and sutured on the sclera of a rabbit, in the areas coinciding with said holes on the retina. A 25 G syringe in which 50 μl of ferrofluid were placed was used for inserting the ferrofluid; this amount was used for each of the holes to be blocked. Making use of the orifices made in the pars plana vitrectomy surgical methodology the syringe was introduced in the eyeball and positioned on the retinal hole for releasing the ferrofluid. Once released, the ferrofluid in each identified hole interacted with the magnetic eye implant sutured in advanced repositioning the retina on the choroids and blocking the hole. The ferrofluid and the magnetic eye implant were removed after 1 week. The results showed 90% of success in the re-application of retina at the end and no strange behavior which allows identifying post-operative recovery anomalies has been identified. The patient experiences no complications.

### Example 8

### Blocking of a Posterior Chamber Retinal Hole Without Using Vitrectomy Pars Plana and Ferrofluid Diluted in Vitreous Humour

50 μl of ferrofluid at a concentration of 200 mg/ml of spherical microparticles (Chemicell. Magnetic microparticles SiMAG. Electronic, 2011), with a diameter of 2 μm, magnetite core, silica coatings and diluted in physiological saline; and a magnetic eye implant of NdFeB with 42 MGOe with silicone elastomer and silica coating with dimensions of 6 mm by an overall thickness of 2 mm, including the magnet and coatings, were used. The implant was placed and sutured on the sclera of a rabbit, in the posterior area of the ocular chamber where the retinal hole is found. A 25 G syringe in which 50 μl of ferrofluid diluted in vitreous humour were placed was used for inserting the ferrofluid. The syringe was introduced in the eyeball and positioned on the retinal hole, in this case a posterior chamber retinal hole, for releasing the ferrofluid. Once released, the ferrofluid interacts with the magnetic eye implant sutured in advanced repositioning the retina on the choroids and blocking the hole. The ferrofluid and the magnetic eye implant were removed after 1 week.

### Example 9

### Blocking of a Posterior Chamber Retinal Hole

50 μl of ferrofluid at a concentration of 50 mg/ml of spherical microparticles with a diameter of 8 μm, spherical porous silicon base with incrustations of ferrous material and diluted in physiological saline; and a magnetic eye implant of NdFeB with 42 MGOe with silicone elastomer and silica coating with dimensions of 6 mm by a thickness of 2 mm all together, including the magnet and coatings, were used. The implant was placed and sutured on the sclera of a rabbit, in the posterior area of the ocular chamber where the retinal hole is found. A 25 G syringe in which 50 μl of ferrofluid diluted in vitreous humour were placed was used for inserting the ferrofluid. The syringe was introduced in the eyeball and positioned on the posterior chamber retinal hole for releasing the ferrofluid. Once released, the ferrofluid interacted with the magnetic eye implant sutured in advanced repositioning the retina on the choroids and blocking the hole. The ferrofluid and the magnetic eye implant were removed after 1 week.

1. An ocular device comprising:
(a) at least one magnetic eye implant comprising
a flat rare earth magnet with magnetization energy between 27852.05 and 35809.78 TA/m,
at least one layer of nickel-copper-nickel (Ni—Cu—Ni) coating and another layer of epoxy or silicone elastomer coating on both faces, and
(b) a ferrofluid comprising a colloidal suspension of microparticles,
wherein said magnetic eye implant and said ferrofluid interact.

2. The ocular device according to claim 1, wherein said rare earth magnet is a NdFeB rare earth magnet.

3. The ocular device according to claim 1, wherein said layer of epoxy or silicone elastomer coating has a thickness comprised between 1 and 1000 μm.

4. The ocular device according to claim 3, wherein said thickness is between 1 and 500 μm.

5. The ocular device according to claim 1, comprising a third layer of silica coating of the eye implant with a thickness comprised between 0.001 and 25 μm, on both faces.

6. The ocular device according to claim 5, wherein said layer of silica is biofunctionalized with amino groups.

7. The ocular device according to claim 1, wherein the magnetic flow in the center of the faces of said implant is comprised between ±1 mT and ±500 mT.

8. The ocular device according to claim 1, wherein said implant is cylindrical-shaped with dimensions of between 3 to 7 mm in diameter by 0.4 to 1.4 mm in height.

9. The ocular device according to claim 8, wherein said cylindrical implant is between 3 and 6 mm in diameter by between 0.5 and 1 mm in height.

10. The ocular device according to claim 8, wherein the magnetization of said implant is localized on the central axis of the cylinder and is comprised in a range of ±185 and ±195 mT.

11. The ocular device according to claim 8, wherein the magnetization of said implant is diametrical and is comprised in a range of ±1 and ±15 mT.

12. The ocular device according to claim 1, wherein it further comprises four suture wedges in said magnetic implant.

13. The ocular device according to claim 1, wherein said ferrofluid has a magnetic response between 10⁻⁶ and 10⁻⁴ Am²/g.

14. The ocular device according to claim 1, wherein said colloidal suspension of the ferrofluid is in physiological saline, vitreous humour or in an alcohol.

15. The ocular device according to claim 1, wherein said microparticles comprise porous silicon and ferrous material, or their oxide derivatives.

16. The ocular device according to claim 1, wherein said microparticles comprise a silicon base with magnetite particle incrustations in the form of flake.

17. The ocular device according to claim 16, wherein said microparticles in the form of flake have a size between 1 and 300 μm at a concentration of between 10 and 250 mg/ml.

18. The ocular device according to claim 17, wherein between 45 and 55% of said microparticles in the form of flake have a size of 100 μm.

19. The ocular device according to claim 16, wherein said microparticles in the form of flake are carriers of pharmacologically acceptable compounds.

20. The ocular device according to claim 1, wherein said microparticles are essentially spherical magnetic microparticles with a core of ferrous material and silica coating, with a diameter of 2 μm and a concentration of 200 mg/ml in the ferrofluid.

21. The ocular device according to claim 20, wherein between 45 and 55% of said spherical microparticles have a size of 2 μm.

22. The ocular device according to claim 1, wherein said microparticles are essentially spherical magnetic microparticles of porous silicon with nanoparticle incrustations, with a diameter between 6 μm and 10 μm.

23. The ocular device according to claim 22, wherein between 45 and 55% of said spherical microparticles have a size of 8 μm.

24. A method of using an ocular device comprising:
a) at least one magnetic eye implant comprising
a flat rare earth magnet with magnetization energy between 27852.05 and 35809.78 TA/m,
at least one layer of nickel-copper-nickel (Ni—Cu—Ni) coating and another layer of epoxy or silicone elastomer coating on both faces, and
b) a ferrofluid comprising a colloidal suspension of microparticles,
wherein said magnetic eye implant and said ferrofluid interact, including the steps of
preparing a surgical system including the at least one magnetic eye implant useful for the operative treatment of pathologies associated with the retina, and
implanting the at least one magnetic eye implant in a patient's eye.

25. The method according to claim 24, wherein the pathology associated with the retina is retinal detachment.

26. The ocular device according to claim 2, wherein said layer of epoxy or silicone elastomer coating has a thickness comprised between 1 and 1000 μm.

27. The ocular device according to claim 17, wherein said microparticles in the form of flake are carriers of pharmacologically acceptable compounds.

28. The ocular device according to claim 18, wherein said microparticles in the form of flake are carriers of pharmacologically acceptable compounds.
